(19) United States
(12) Patent Application Publication
(10) Pub. No.: US 2026/0350798 A1
(43) Pub. Date: Nov. 5, 2026

(54) Adaptive Disentangled Multi-Modal Learning With Patient-Aware Fusion For Intracranial Aneurysm Segmentation And Rupture Risk Prediction

(71) Applicant: REGENTS OF THE UNIVERSITY OF MICHIGAN, Ann Arbor, MI (US)

(72) Inventors: Muhammad Irfan, Grand Blanc, MI (US); Khalid Mahmood Malik, Rochester Hills, MI (US)

(21) Appl. No.: 19/668,799

(22) Filed: May 5, 2026

Related U.S. Application Data
(60) Provisional application No. 63/800,179, filed on May 5, 2025.

(51) Int. Cl. G06T 7/00; G16H 10/60; G16H 30/40; G16H 50/30

(52) U.S. Cl. CPC G06T 7/0012; G16H 10/60; G16H 30/40; G16H 50/30; G06T 2207/10116; G06T 2207/30016

(57) ABSTRACT

A computer-implemented method, system, and computer-readable media for adaptive multimodal learning for intracranial aneurysm segmentation and rupture risk prediction. The disclosed techniques extract features from medical imaging data and clinical and radiological data, align the features across modalities, fuse the aligned features, and classify an aneurysm rupture probability based on the fused features. In some aspects, features may be decomposed into modality-specific representations and task-common representations, with orthogonality constraints enforced to reduce feature redundancy. Contrastive alignment may pull task-common representations from the same patient closer in a latent space while pushing apart representations from different patients. Adaptive fusion may be performed using Feature-wise Linear Modulation (FILM) conditioning for rupture severity prediction and Adaptive Instance Normalization (AdaIN) conditioning for segmentation decoding. A multi-task objective combining segmentation loss, classification loss, orthogonality regularization, and contrastive alignment loss may jointly supervise the learning process.

### CROSS REFERENCE TO RELATED APPLICATIONS

This application claims priority to U.S. Provisional Patent Application No. 63/800,179, titled “Adaptive Learning for Multi-Modal Intracranial Aneurysm Rupture Criticality Analysis,” filed May 5, 2025, which is hereby incorporated by reference in its entirety.

### TECHNICAL FIELD

The present disclosure relates to computer-implemented methods and systems for medical image analysis. More particularly, the disclosure relates to adaptive, disentangled multi-modal learning frameworks for intracranial aneurysm segmentation and rupture risk prediction using imaging and clinical data.

### BACKGROUND

Conventional assessments of cerebral aneurysm rupture risk rely upon clinical scoring systems. However, such systems have limitations in terms of predictive accuracy. While other image-based techniques have advanced aneurysm detection and characterization methods, they often lack integration with patient-specific clinical and radiological information, which is useful for personalized aneurysmal rupture risk assessment. Moreover, many previous systems rely on a single image modality and may not effectively capture morphological and hemodynamic features relevant to aneurysm rupture risk predictions. This image-based reliance for aneurysm rupture prediction fails to capture clinical aspects, thus limiting clinical interpretability. Integrating heterogeneous data types presents technical challenges, including the difficulty of aligning features across modalities that have different statistical properties, dimensionalities, and semantic representations. These technical challenges are compounded by limitations in multimodal learning approaches.

Furthermore, recent advances in multimodal learning have attempted to integrate heterogeneous data sources. However, existing approaches may rely on static fusion strategies or concatenation, which fail to capture patient-specific variability and modality-dependent relevance. Additionally, conventional approaches do not disentangle modality-specific and shared representations, leading to semantic leakage, poor interpretability, and reduced generalization performance. When modality-specific and shared feature representations are not separated, the resulting feature spaces may contain redundant information that degrades computational efficiency and model performance. Furthermore, existing systems treat segmentation and rupture risk prediction as independent tasks, failing to leverage shared anatomical and clinical representations that could improve both spatial localization and severity estimation. This independent treatment of related tasks results in duplicated computation. Accordingly, for at least the reasons above, there remains a need for unified frameworks that can perform segmentation and risk prediction while adaptively integrating multimodal information in a patient-aware manner.

### BRIEF SUMMARY OF THE INVENTION

In some aspects, the present techniques described herein relate to a computer-implemented method for multi-modal adaptive learning including: (i) extracting, by one or more processors, features from medical imaging data and clinical and radiological data; (ii) aligning, by the one or more processors, the features from the medical imaging data with the features from the clinical and radiological data; (iii) fusing, by the one or more processors, the features from the medical imaging data and the clinical and radiological data; and (iv) classifying, by the one or more processors, an aneurysm rupture probability based on the aligned and fused features from the medical imaging data and the clinical and radiological data.

In some aspects, the techniques described herein relate to a computing system including: one or more processors; and one or more memories storing computer-executable instructions that cause the one or more processors to at least: (i) extract features from medical imaging data and clinical and radiological data; (ii) align the features from the medical imaging data with the features from the clinical and radiological data; (iii) fuse the features from the medical imaging data and the clinical and radiological data; and (iv) classify an aneurysm rupture probability based on the aligned and fused features from the medical imaging data and the clinical and radiological data.

In some aspects, the techniques described herein relate to one or more non-transitory computer-readable media storing processor-executable instructions that, when executed by one or more processors, cause the one or more processors to perform operations including: (i) extracting features from medical imaging data and clinical and radiological data; (ii) aligning the features from the medical imaging data with the features from the clinical and radiological data; (iii) fusing the features from the medical imaging data and the clinical and radiological data; and (iv) classifying an aneurysm rupture probability based on the aligned and fused features from the medical imaging data and the clinical and radiological data.

### BRIEF DESCRIPTION OF THE DRAWINGS

The skilled artisan will understand that the figures described herein are included for purposes of illustration and do not limit the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like primary characters throughout the various drawings generally refer to functionally similar or structurally similar components.

FIG. 1 is a simplified block diagram of an example system for predicting aneurysm rupture risk.

FIG. 2A is a block and logic flow diagram of example clinical data embedding, in accordance with the present disclosure.

FIG. 2B is a block and logic flow diagram of an example token-based transformer clinical encoder method, in accordance with the present disclosure.

FIG. 3A is a block and logic flow diagram of example Siamese attention-driven features, in accordance with the present disclosure.

FIG. 3B is a block and logic flow diagram of an example hierarchical transformer image encoder method, in accordance with the present disclosure.

FIG. 4A is a block and logic flow diagram of example constrained modality-specific features, in accordance with the present disclosure.

FIG. 4B is a block and logic flow diagram of an example adaptive disentangled multi-modal learning method with patient-aware fusion, in accordance with the present disclosure.

FIG. 5 is a flow diagram depicting an example method for multi-modal prediction of aneurysm rupture risk.

FIG. 6 is a visualization of task-specific and common feature representations for segmentation and severity prediction tasks across image and clinical modalities, in accordance with the present disclosure.

### DETAILED DESCRIPTION

The present techniques generally involve extracting features from both medical imaging data and clinical and radiological data, aligning these features across modalities, fusing the aligned features into a unified representation, and classifying an aneurysm rupture probability based on the aligned and fused features. The present disclosure aims to reduce problems with conventional techniques (e.g., as described in the Background section) by providing techniques for multi-modal adaptive learning. The present techniques may include advantages to the field of medical diagnostics, machine learning, multi-modal knowledge exchange, as well as clinical decision support systems, evidence-based medicine, and medical image analysis, thereby enhancing diagnostic accuracy and predictive modeling in both cardiovascular and cerebrovascular domains.

With respect to feature extraction, the present techniques may pre-process unstructured data and other formats, structures, and physical realizations (e.g., complex and unformatted medical history, clinicians' handwritten notes, contrasted imaging, etc.). For clinical data, named entity recognition (NER) may transform unstructured clinical text into structured, semantically meaningful representations, while normalization techniques such as min-max normalization transform features to a predetermined range (e.g., [0, 1]). This preprocessing may reduce sensitivity to variations in scale across modalities and improve convergence speed during training by reducing internal covariance shift. Linear projection may then map the normalized clinical data summaries into a standardized, high-dimensional numerical space, enabling integration across modalities and ensuring consistent data compatibility for relationship analysis between clinical risk factors and anatomical characteristics.

For medical imaging data, the present techniques may employ patch-level inference and Siamese attention mechanisms to extract features from image patches. This approach may ensure that patches with similar anatomical structures (e.g., those showing a potential aneurysm region) are mapped closely together in the feature space while distinguishing them from patches with normal anatomy. Multi-instance learning may further partition patches into positive and negative bags based on whether they contain pathologically relevant information.

With respect to feature alignment, the present techniques may employ transformer encoders with self-attention mechanisms to align the extracted clinical features with image-derived features. This alignment may facilitate a comprehensive multi-modal analysis of aneurysm rupture risk by correlating image-derived characteristics (e.g., aneurysm size, shape, and location) with clinical attributes (e.g., patient age, risk factors, or medical history). The fusion of clinical and imaging information into a high-dimensional vector space may enable greater accuracy and interpretability compared to single-modality approaches.

With respect to feature fusion, the present techniques may employ bottleneck fusion with cross-modal attention mechanisms to combine common features from both modalities while preserving modality-specific information. Adversarial projection with a modality discriminator may enforce alignment between shared features and distinguishable modality-specific features, promoting the extraction of modality-invariant representations and enhancing cross-modal alignment. Cosine similarity constraints may further ensure that local (modality-specific) features remain orthogonal to global (shared) features, reducing redundancy while maintaining independent and discriminative representations.

By integrating multi-modal data sources in a single learning framework through these extraction, alignment, and fusion operations, the present techniques may improve processing speeds, diagnostic accuracy, and model interpretability. The techniques may enable faster convergence during training by normalizing input data and hidden activations, reduce sensitivity to variations in scale across different modalities, and produce robust, discriminative embeddings that support enhanced clinical decision-making. In this context, the techniques may be directed to improvements in efficiency, precision in aligning imaging and clinical features, and overall better generalization performance when predicting aneurysm rupture risk, thereby aiding in interpretability.

To address the technical challenges of semantic leakage and feature redundancy in multi-modal learning systems, the present techniques may decompose input data into modality-specific and shared latent representations. For example, imaging data and clinical data may each be processed through dedicated encoders that produce separate latent codes for task-specific information (e.g., severity-relevant features, segmentation-relevant features) and task-common information shared across modalities. Shared representations may be aligned using contrastive learning to enforce semantic consistency across modalities, while orthogonality constraints may be applied to ensure that modality-specific features capture independent information from the shared representations, thereby reducing feature redundancy and semantic leakage between feature subspaces. This structured decomposition may improve computational efficiency by enabling each downstream task to operate on a targeted subset of the latent space rather than processing redundant, entangled feature representations.

Rather than relying on static fusion strategies that assign uniform importance to all modalities, the present techniques may employ adaptive fusion mechanisms that integrate aligned clinical and imaging representations using a patient-specific conditioning signal. The conditioning signal may dynamically modulate modality-specific features to regulate their relative contributions during fusion, thereby enabling patient-aware integration of multimodal information. For example, Feature-wise Linear Modulation (FILM) conditioning, Adaptive Instance Normalization (AdaIN), or other feature-wise modulation techniques may generate adaptive scaling and shifting parameters from a conditioning vector that aggregates severity-relevant features from both modalities together with shared cross-modal representations, producing a patient-aware modulated representation for rupture severity prediction. Additionally or alternatively, AdaIN or other adaptive normalization mechanisms may condition a hierarchical segmentation decoder on segmentation-relevant features from both imaging and clinical modalities, enabling anatomically consistent spatial reconstruction that adapts to patient-specific clinical and imaging context.

The present techniques may employ a multi-task learning framework that jointly performs aneurysm segmentation and rupture severity prediction using shared and modality-specific feature representations. By jointly optimizing both tasks, the system may enable shared learning of complementary features, where anatomical and morphological information learned for segmentation informs risk prediction, and global clinical context enhances spatial delineation. The multi-task learning process may be guided by a composite objective function that balances segmentation accuracy and classification performance, and may include regularization terms that promote disentanglement and cross-modal consistency. This joint optimization may reduce duplicated computation compared to systems that treat segmentation and risk prediction as independent tasks, while enabling complementary gradient signals from both tasks to improve the learned representations.

Additional advantages of the present techniques over conventional approaches will be appreciated throughout this disclosure by one of ordinary skill in the art. The various concepts and techniques introduced above and discussed in further detail below may be implemented in numerous ways, and the described techniques are not limited to any particular manner of implementation. Examples of implementations are provided below for illustrative purposes.

### Example Computing System

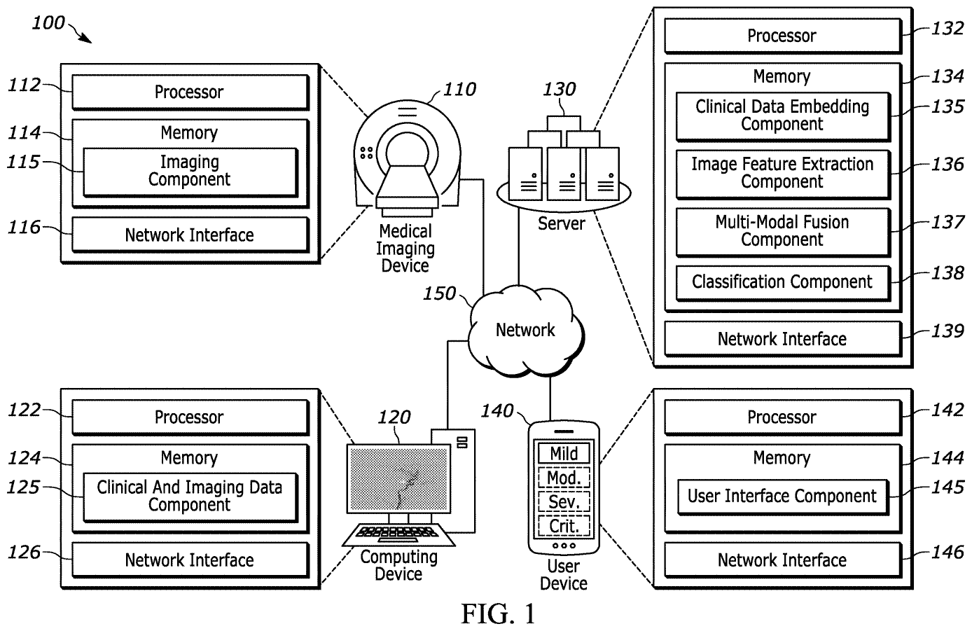

FIG. 1 generally depicts an example computing system 100 for classifying an aneurysm rupture probability/criticality. The computing system 100 may include a medical imaging device 110, computing device 120, server 130, user device 140, and a network 150. The computing system 100 is not limited by the specific components, devices, and/or examples described herein but may include any suitable number, type, and architectural arrangement of components, devices, and/or examples in accordance with the present disclosure.

### Imaging Device

The medical imaging device 110 may generally capture medical imaging data (e.g., for aneurysm detection and analysis). In the example of FIG. 1, the medical imaging device 110 includes a processor 112, a memory 114, and a network interface 116. In such examples, the processor 112 may perform real-time image acquisition, processing, and/or reconstruction algorithms (e.g., Fourier transformations, parallel processing, transformer-based methods, and/or other methods to convert raw sensor data into diagnostically useful images). The memory 114 includes an imaging component 115 storing computer-readable instructions and/or acquired medical imaging data (e.g., rotational DSA images, MR image slices, CT scan data, etc.) for further processing and analysis. That is, the memory 114 may be and/or include any suitable storage device for storing medical imaging data (e.g., rotational DSA images). The imaging component 115 may also include computer-readable instructions (e.g., software) for a graphical user interface (GUI) enabling technicians (e.g., clinicians) to interact with, review, analyze, etc. the medical imaging data. Furthermore, the network interface 116 may be configured to facilitate communication with the network 150 and other devices and/or components of the example computing system 100, and/or with external networks (e.g., for transmitting medical imaging data for additional processing, remote diagnostics, data archiving, etc.). The processor 112, memory 114, and network interface 116 may be substantially similar to the processor 122, memory 124, and network interface 126 described in further detail below.

In one example, the medical imaging device 110 may be and/or include an X-ray machine, a magnetic resonance imaging (MRI) machine, a magnetic resonance angiography (MRA) machine, a digital subtraction angiography (DSA) system, or any other suitable medical imaging device now known or later developed. The medical imaging device 110 may capture contrast-enhanced images (e.g., rotational DSA) enabling visualization of vascular structures and, in turn, facilitating detailed assessment of vascular pathologies (e.g., aneurysms). The medical imaging device 110 may include a primary magnet assembly (e.g., in MRI/MRA examples) and/or an X-ray tube (e.g., in X-ray examples) which, together with gradient coils (e.g., for MRI/MRA) or other spatial encoding mechanisms, enables generation, using the processor 112, of spatially encoded medical imaging data. In examples with contrast (e.g., DSA), the medical imaging device 110 may acquire time-series image data highlighting dynamic blood flow, distinguishing vascular structures from surrounding tissues. In another example, the medical imaging device 110 may dynamically image sequences capturing three-dimensional medical imaging data (e.g., rotational acquisitions in DSA) of a pathology (e.g., aneurysm) localization and/or characterization.

In examples involving vascular assessments (e.g., aneurysm rupture prediction), the imaging component 115 may include computer-executable instructions for optimizing the vision for rotational DSA imaging, optimizing the visualization of blood vessels, and/or to otherwise highlight vascular features for diagnostic, analytical, and/or training purposes.

In further examples, the imaging component 115 may be compatible across a plurality of medical imaging devices 110 (e.g., X-ray, CT, CTA, MRI, MRA, DSA, etc.). It should be understood that the example of FIG. 1 may be adapted for a variety of diagnostic clinical applications (e.g., other than aneurysm rupture risk) to improve diagnostic accuracy and clinical interpretability. The present disclosure may be compatible across a broad range of imaging modalities (e.g., X-ray, MRI, MRA, DSA, etc.). This compatibility advantageously renders the present disclosure suitable for a wide variety of diagnostic clinical applications, such as systems that incorporate multi-modal data fusion (e.g., merging imaging data with associated clinical records) for enhanced predictive analysis of conditions such as intracranial aneurysm rupture, and other systems to improve overall diagnostic accuracy and aid clinical decision-making.

### Computing Device

The computing device 120 may be an individual computing device, a group (e.g., cluster) of multiple computing devices, or another suitable type or system of computing resources. For example, the computing device 120 may be any suitable computing device (e.g., a laptop, desktop computer, workstation, embedded computing system, microcontroller, etc.). In some examples, components of the computing device 120 may be embodied by virtual instances using a cloud-based virtualization service, in which case the computing device 120 may reside in a remote data center (e.g., somewhere different from the other components of the example computing system 100). In the example of FIG. 1, the computing device 120 includes a processor 122, a memory 124, and a network interface 126. The processor 122 may include any suitable number of processors and processor types, such as central processing units (CPUs), graphics processing units (GPUs), tensor processing units (TPUs), field-programmable gate arrays (FPGAs), or application-specific integrated circuits (ASICs), and generally provides hardware configured to execute instructions stored in the memory 124. The memory 124 may include volatile and non-volatile memory types and may store an operating system, application modules, executable instructions, machine learning models, and other data, all of which represent non-transitory computer-readable media. The memory 124 may include computer-executable instructions (e.g., software, program, code, script, package, application, etc.) providing a GUI for receiving, transmitting, accessing, etc. medical imaging and clinical data (e.g., imaging and clinical data component 125). The network interface 126 may comprise hardware and/or software components enabling the computing device 120 to communicate with other components of the network 150 according to any suitable communication protocol (e.g., Digital Imaging and Communications in Medicine (DICOM), Health Level Seven (HL7), AES-256 encryption, etc.)

### Server

The server 130 may be an individual server, a group of servers, or another suitable type of server, and, for example, may be any appropriate server device (e.g., rack server, tower server, blade server, mainframe, cloud server, virtual server, distributed server). In some examples, components of the server 130 may be embodied by virtual instances hosted in a remote data center (e.g., a location different from the other components of the computing system 100). In the example of FIG. 1, the server 130 includes a processor 132, a memory 134, and a network interface 139. The memory 134 may include computer-executable instructions for embedding clinical data (e.g., via clinical data embedding component 135), extracting features from medical imaging data (e.g., via image feature extraction component 136), multi-modal feature fusion and adaptive integration (e.g., via multi-modal fusion component 137), classifying aneurysm rupture probability (e.g., via classification component 138), and/or otherwise processing the medical imaging and clinical data in accordance with the present disclosure (e.g., via disentangled representation learning, contrastive alignment, orthogonality regularization, adaptive conditioning, multi-task loss optimization, etc.). In some embodiments, the image feature extraction component 136 may implement a hierarchical transformer image encoder that partitions medical imaging data into patch tokens and processes them through stacked multi-head self-attention layers to produce disentangled latent representations. In some embodiments, the multi-modal fusion component 137 may implement adaptive fusion using Feature-wise Linear Modulation (FILM) conditioning for rupture severity prediction and Adaptive Instance Normalization (AdaIN) conditioning for segmentation decoding. In some embodiments, the image feature extraction component 136 may alternatively implement a Siamese attention mechanism with multi-instance learning to extract patch-level features, and the multi-modal fusion component 137 may alternatively implement constrained modality-specific feature extraction using bottleneck fusion with adversarial projection and a modality discriminator.

### User Device

The user device 140 may be an individual user device, a group of multiple user devices, or another suitable type of user device. For example, the user device 140 may be any suitable device (e.g., smartphone, tablet, 2-in-1 device, smartwatch, e-reader, smart glasses, IoT-enabled device, etc.). In the example of FIG. 1, the user device 140 includes a processor 142, a memory 144, and a network interface 146. The memory 144 may include computer-executable instructions for receiving and/or accessing prediction outcomes. In the example of FIG. 1, the memory 144 includes a graphical user interface (GUI) (e.g., user interface component 145) for displaying to a user (e.g., clinician, patient, representative, etc.) an aneurysm rupture risk (e.g., mild, moderate, severe, critical). In some examples, the user interface component 145 may include additional aspects (AI agent integration, interpretability of results, further analysis, reports, notifications, etc.).

### Network

The network 150 may include wired and/or wireless communication networks such as cellular networks (e.g., 5G®, 4G LTE®, 3G®), Wi-Fi® networks (e.g., IEEE 802.11 standards), microwave access networks (e.g., WiMAX®), or any other suitable wide area networks (WAN), local area networks (LAN), or personal area networks (PAN). The network 150 may consist of a single communication network or multiple networks of different types (e.g., separate networks for communications between the medical imaging device 110, computing device 120, and the server 130 or between the computing device 120 and the user device 140), and may include tens, hundreds, or thousands of networks.

### Example Aneurysm Rupture Risk Prediction

In operation, the computing system 100 may classify an aneurysm rupture risk based on clinical data (e.g., textual clinical and radiology notes) and medical imaging data (e.g., captured via medical imaging device 110). The medical imaging device 110, using the processor 112, may execute computer-readable instructions for acquiring medical imaging data (e.g., using application-specific sensors to obtain rotational DSA and/or MRA images of a potential aneurysm rupture). The computer-readable instructions may be configured to transmit (e.g., via network interface 116) the data to the computing device 120 and/or server 130 (e.g., for review, further analysis, to optimize processing resources, etc.). It should be understood that, in some examples, the computing system 100 may not include the medical imaging device 110 and in such cases, may otherwise acquire medical imaging data from any other suitable source (e.g., synthetically generated medical imaging data, cloud-based hosting service, healthcare provider-specific inputs, etc.).

The computing device 120, using the processor 122, may execute computer-readable instructions (e.g., clinical and imaging data component 125) to receive, store, transmit, and/or otherwise access medical imaging data and clinical data (e.g., medical records of a patient) corresponding to the medical imaging data. The computer-readable instructions may be configured to transmit (e.g., via network interface 126) the medical imaging data and/or clinical data to the server 130 (e.g., for analysis, machine learning, training, inference, etc.).

The server 130, using processor 132, may execute computer-readable instructions for encoding clinical variables as learnable tokens via a transformer clinical encoder, extracting imaging features (e.g., aneurysm size, shape, location, etc.) via a hierarchical transformer image encoder, decomposing modality-specific and task-common latent representations via dedicated projection heads, aligning common representations across modalities via contrastive learning, conditioning rupture severity prediction via FILM modulation, conditioning segmentation decoding via AdaIN modulation, and computing multi-task losses including segmentation, classification, orthogonality, and contrastive alignment terms, etc. In some embodiments, the server 130 may alternatively execute computer-readable instructions for clinical patch-level inference, clinical data embedding (e.g., age, risk factors, medical history, etc.), aligning global and local features (e.g., using a transformer encoder and adversarial projection) to extract modality-specific and shared features (e.g., using a modality discriminator), calculating losses based on logarithmic evaluations to refine the feature fusion, aligning features using a self-attention mechanism, and fusing the aligned features through a bottleneck to direct cross-modal attention.

The computer-readable instructions may be configured to generate a classification (e.g., using classification component 138) and transmit (e.g., via network interface 139) the classification to the user device 140. The user device 140 may execute, using processor 142, computer-readable instructions (e.g., user interface component 145) to receive and/or display the classification (e.g., aneurysm rupture probability). In sum, the computing system 100 may extract features from both medical imaging data and clinical data, aligns the features across modalities, fuses the aligned features using adaptive conditioning mechanisms, and classifies an aneurysm rupture risk probability. In some embodiments, the computing system 100 may additionally generate an aneurysm segmentation map based on the fused features.

It should be understood that, in some examples, the computing system 100 may not include certain devices (e.g., the user device 140) and in such cases provide a clinician (e.g., neurosurgeon, radiologist, other healthcare professional), patient, and/or any other interested party, with a classification by any suitable technique (e.g., display on a healthcare provider-specific charting system, save to a 3rd-party charting system, save to a 3rd-party database, audibly transcribe the classification, trigger an AI agent workflow, etc.) to enable informed treatment.

### Example Clinical Data Embedding and Encoding

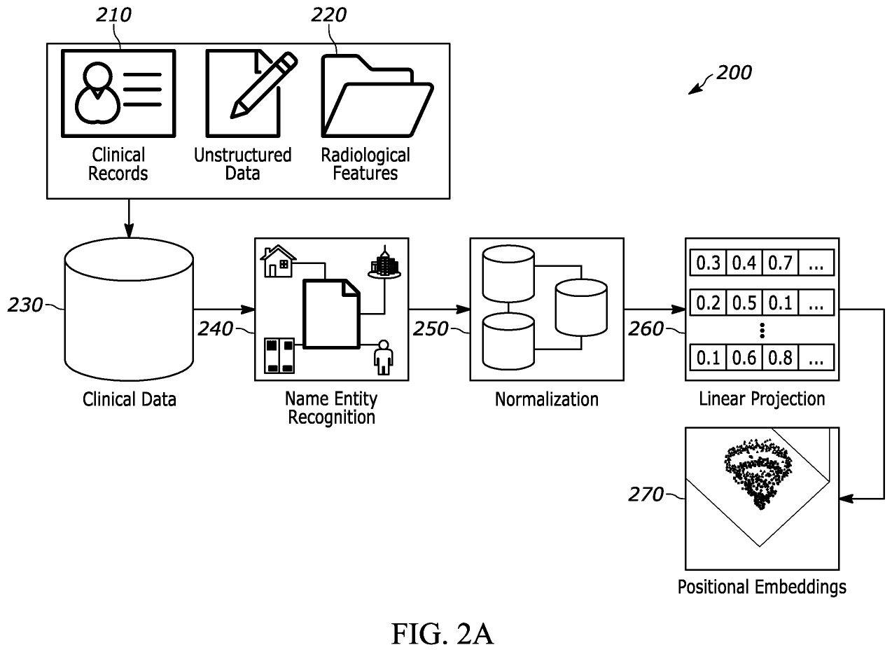

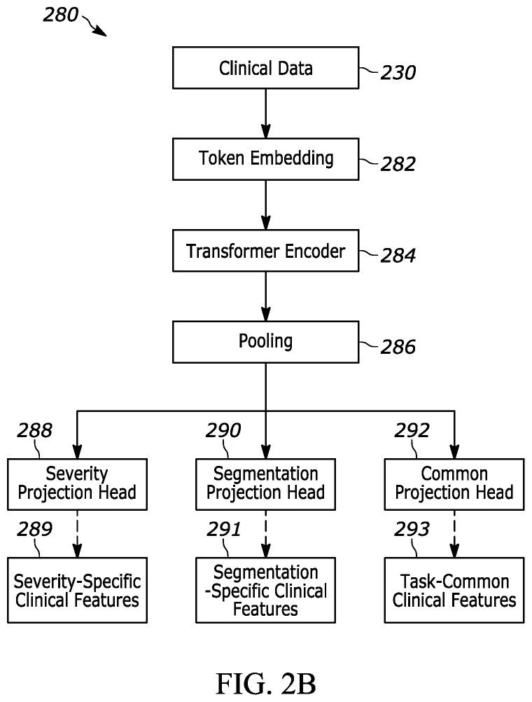

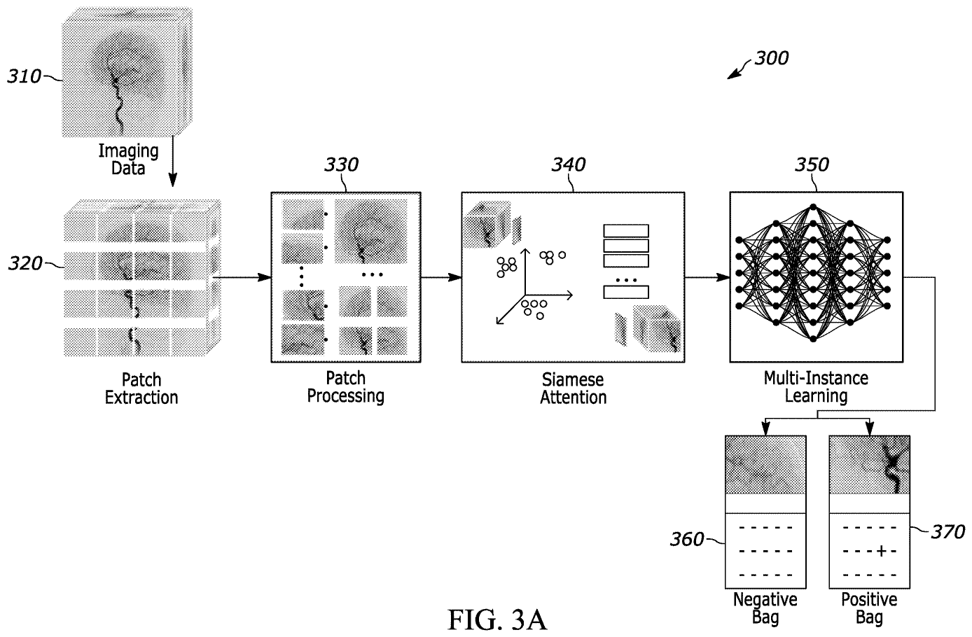

FIG. 2A generally depicts an example clinical data embedding block and logic flowchart 200 (e.g., representing functionality of clinical data embedding component 135 as described above, with respect to FIG. 1). The flowchart 200 includes clinical records 210, radiological features 220, clinical data 230, named entity recognition (NER) 240, normalization 250, linear projection 260, and positional embeddings 270. The clinical data embedding flowchart 200 includes the clinical data 230 (e.g., clinical records 210 and radiological features 220, which may be and/or include unstructured data), NER 240 and normalization 250 to process values:

$${\{ {y}_{i} \}}_{i = 1}^{n}$$

and embeds this data using linear projection 260 as:

A=[D(y₁),D(y₂), . . . ,D(yₙ)]+B

where A∈R^{n×p} corresponds to the clinical information, D is the linear transformation of R^{1×p}, and B is a learned position encoding to maintain order and sequential information. The clinical data embedding flowchart 200 illustrates mapping the normalized clinical data into a high-dimensional latent space of positional embeddings from linear projection 260.

Clinical data 230 generally includes clinical records 210 (e.g., medical records including age, medical history, lifestyle factors, patient demographics, risk factors, etc.) and/or radiological data including radiological features 220 (e.g., aneurysm size, shape, vascular geometry from rotational DSA, etc.). Advantageously, present techniques may pre-process unstructured data and other formats, structures, physical realizations (e.g., complex and unformatted medical history, clinicians' handwritten notes, contrasted imaging, etc.) to enable efficient use of resources (GPU, CPU, TPU, etc.) given constraints of the environment (e.g., at a hospital, an ambulatory site, data center via an API, etc.). The clinical data 230, including unstructured text, is processed to identify, extract, and classify entities (e.g., patient demographics, medical history elements, risk factors such as hypertension or smoking, anatomical locations, time expressions, etc.). As used herein, the terms “clinical data” and “clinical and radiological data” may both refer to patient-related information including clinical records (e.g., age, medical history, risk factors) and radiological features (e.g., aneurysm size, shape, vascular geometry).

The NER 240, or other natural language processing techniques for extracting structured information from unstructured clinical text, converts raw, heterogeneous textual data (e.g., clinical notes, radiology reports, medical history, etc.) into structured, semantically meaningful embeddings. By extracting these named entities, the NER 240 normalizes the clinical text by locating beneficial pieces of information, advantageously supporting consistency in feature embedding.

The NER enables efficient and reliable extraction of structured information from unstructured clinical narratives. When fused with high-dimensional imaging features from modalities such as DSA, MRA, or MRI, these structured text embeddings provide complementary context that can improve the predictive power of the multi-instance learning framework.

By converting clinical notes via NER 240 into detailed attribute-level embeddings, the system is better able to capture subtle details (e.g., specific anatomical terms, risk-factor identifiers, clinical codes, etc.) that might otherwise be lost when dealing with raw text. This trains the transformer encoder's self-attention mechanisms to correlate precisely the relevant entities from clinical inputs with corresponding imaging features. Integrating the present techniques in a larger hemodynamic and clinical decision-making pipeline reduces manual curation and facilitates scalable processing of clinical reports as they are generated, seamlessly incorporating structured textual data into a multi-modal feature space that leverages both global (shared) and local (modality-specific) representations. In sum, the NER 240 transforms unstructured clinical text into a structured, semantically rich representation. This enriched representation improves the integration of clinical data with complex imaging features (e.g., derived from DSA, MRA, MRI, etc.).

The normalization 250 algorithmically summarizes the clinical data 230 into concise, semantically meaningful representations using an appropriate technique (e.g., natural language processing (NLP), including large language models (LLMs), semantic analysis, quality assessment, etc.).

Normalization 250 may generally include activations, applied machine learning, standardizing inputs, and/or intermediate representations. Normalization 250 (e.g., feature scaling) may ensure that one or more input features (e.g., a clinical parameter such as age, blood pressure, or risk factor scores, and imaging-derived parameters such as aneurysm size, shape, and location) are rescaled to share common statistical properties (e.g., a similar range, mean, variance, etc.). In one example, a min-max normalization may transform features to a predetermined range (e.g., [0, 1]), to prevent features measured in different units (e.g., centimeters vs. nanometers) from disproportionately influencing the learning process. This preprocessing advantageously reduces the sensitivity to variations in scale across modalities and improves convergence speed during training by reducing internal covariance shift.

In some examples, activation normalization techniques (e.g., Batch Normalization (BatchNorm), Layer Normalization (LayerNorm)) and/or adaptive normalization used in transformers (e.g., FixNorm or ScaleNorm) may be employed within a neural network (e.g., in the hidden layers). The transformer encoder may generally process imaging features and clinical embeddings and use activation normalization by stabilizing the distribution of hidden activations. This stabilization accelerates the training process (e.g., smoothing the optimization landscape) and helps prevent overfitting, thereby enhancing the generalization of the model to unseen data. For instance, in the multi-modal encoder layers that integrate features using self-attention mechanisms, applying LayerNorm across features ensures that one or more attention heads operate on inputs with consistent statistics, regardless of the differences between imaging and clinical domains.

Moreover, normalization within adversarial projection, which enforces modality-invariant representations and separate modality-specific features, allows for improved consistency when aligning and contrasting fused features. By normalizing both the shared and the specific components of the features through the techniques described herein, the training process achieves a balanced representation that supports cross-modal learning. Normalization may increase the effective learning rate and mitigates the issues associated with deep network training (e.g., vanishing/exploding gradients). Such benefits will be appreciated for processing complex datasets comprising multiple imaging modalities (e.g., MRI, MRA, CT, CTA, X-Ray, DSA) alongside heterogeneously scaled clinical data. In some embodiments, layer normalization within the transformer encoder may stabilize the distribution of hidden activations across self-attention and feed-forward layers, allowing for improved consistency when aligning and integrating clinical token representations with image-derived features. By normalizing both the common and the modality-specific components of the latent representations through the techniques described herein, the training process may achieve a balanced disentangled representation that supports robust cross-modal learning.

Additionally, the combination of data normalization and activation normalization may support implementation of adaptive normalization (e.g., cosine similarity constraints within the loss functions). This enforces orthogonality between modality-specific feature spaces and may help ensure the dynamic weighting through attention mechanisms remains effective. By maintaining consistency across input batches and hidden layers, the system effectively performs multi-instance learning and cross-modal fusion, driving better convergence during training and yielding more accurate aneurysm rupture probability predictions. In some embodiments, the combination of data normalization and activation normalization may additionally support implementation of adaptive normalization mechanisms, including AdaIN in the segmentation decoder and FILM conditioning in the severity prediction path. Orthogonality regularization may enforce that modality-specific subspaces capture non-overlapping information relative to the shared latent space, while contrastive alignment via Information Noise-Contrastive Estimation (InfoNCE) loss may ensure that common representations from imaging and clinical modalities are semantically aligned. By maintaining consistency across input batches and hidden layers, the system may effectively perform disentangled multimodal learning and adaptive cross-modal fusion, driving better convergence during training and yielding more accurate aneurysm rupture probability predictions.

The incorporation of both data normalization and activation normalization in the present multi-modal aneurysm rupture prediction techniques serves to increase training convergence speed, reduce sensitivity to input scale variations, mitigate overfitting, and improve generalization in complex clinical environments. This robust normalization framework enables the multi-modal data (e.g., despite its diversity, scale differences, and variability) to be effectively integrated and processed to provide an accurate, explainable prediction of aneurysm rupture criticality.

Advantageously, the linear projection 260 maps the clinical data summaries into a standardized, high-dimensional numerical space, enabling integration across modalities and ensuring consistent data compatibility for relationship analysis between clinical risk factors and/or anatomical characteristics.

In some examples, clinical records 210 may include unstructured data like handwritten notes (e.g., “65-year-old with hypertension and a history of smoking”). The clinical records 210 may be converted into vectors that capture the predictive relevance. Similarly, the clinical records 210 may include radiological features (e.g., “7 mm irregularly shaped aneurysm”). The radiological features may be encoded with suitable spatial and geometric precision and/or notation.

In other examples, the clinical data embedding flowchart 200 may include converting qualitative clinical details such as age and/or gender (e.g., binary-coded gender where “1” represents male and “0” represents female) into concise word-level embeddings. Advantageously, this enables a transformer encoder to align and fuse these clinical features with image-derived features, thereby facilitating a comprehensive multi-modal analysis of aneurysm rupture risk.

FIG. 2B depicts an example token-based transformer clinical encoder method 280 (e.g., representing additional and/or alternative functionality of clinical data embedding component 135 as described above, with respect to FIG. 1). Clinical data 230 is received by a token embedding operation 282 that maps each structured clinical variable into a learnable vector of fixed dimension, forming an input token sequence. For example, the token embedding operation 282 may map each clinical variable into a vector of fixed dimension do, forming the input sequence:

X_{clin}∈ [symbol] ^{N}^{_{c}}^{×d}^{ₒ}

where N_{c} denotes the number of clinical features. This token-based representation may preserve the identity of each clinical attribute (e.g., age, blood pressure, hypertension status, aneurysm morphology) while enabling the transformer to model dependencies across variables through self-attention.

The token embedding operation 282 provides the input token sequence to a transformer encoder 284 that may process the token sequence through stacked self-attention and feed-forward layers, or other suitable sequence modeling operations, capturing inter-variable correlations among factors such as demographic, hemodynamic, and morphological factors. A learnable class token or global pooling operation may be applied to obtain a compact clinical representation, followed by a nonlinear projection, yielding:

$${h}_{clin} = {Ψ}_{cnt}^{clin} ( Pool ( {X}_{clin} ) ) ∈ {\text{?}}^{{d}_{h}}$$

In some examples, the transformer encoder 284 provides output to a pooling operation 286 that applies global average pooling, a learnable class token to obtain a compact clinical representation, or other suitable aggregation technique. The pooling operation 286 provides output to three parallel projection heads: a severity projection head 288 that produces severity-specific clinical features 289, a segmentation projection head 290 that produces segmentation-specific clinical features 291, and a common projection head 292 that produces task-common clinical features 293 shared with the image modality. Although the projection heads are depicted as operating in parallel in FIG. 2B, it should be understood that additional and/or alternative configurations may also be used (e.g., sequential processing, selective activation of one or more projection heads, or other suitable arrangements).

By processing each clinical variable as an independent learnable token, the transformer encoder 284 may learn to correlate the relevant clinical attributes with one another and with corresponding imaging features. The three dedicated projection heads further decompose the pooled clinical representation into task-specific latent factors. The severity projection head 288 produces severity-specific clinical features 289:

$${s}_{sev}^{clin} ∈ {\text{?}}^{{d}_{s}}$$

for severity-relevant clinical cues such as patient age, hypertension status, and smoking history. The segmentation projection head 290 produces segmentation-specific clinical features 291:

$${s}_{seg}^{clin} ∈ {\text{?}}^{{d}_{s}}$$

for segmentation-relevant clinical cues such as aneurysm location descriptors and vascular anatomy indicators. The common projection head 292 produces task-common clinical features 293:

$${z}_{com}^{clin} ∈ {\text{?}}^{{d}_{z}}$$

for task-common representations shared with the image modality. This disentanglement may enable the network to isolate patient-level risk factors governing rupture severity from structural descriptors relevant to anatomical segmentation, while preserving shared information that contributes to both predictions.

### Example Image Feature Extraction

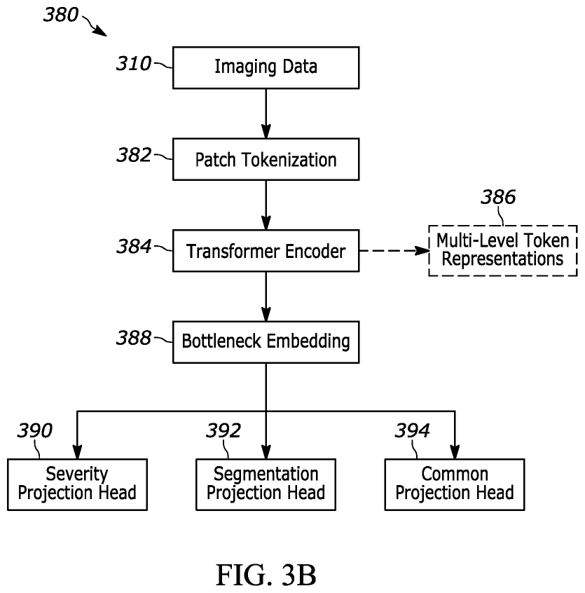

FIG. 3A generally depicts an example Siamese attention driven features (SADF) block and logic flowchart 300. The flowchart 300 includes imaging data 310, patch extraction 320, patch processing 330, Siamese attention 340, multi-instance learning (MIL) 350, negative bag 360, and positive bag 370. Generally, the Siamese attention driven features (SADF) flowchart 300 depicts how raw imaging data 310 is systematically processed using patch extraction 320, patch processing 330, and then refined into a compact feature representation by Siamese attention 340 (e.g., using a Siamese attention technique). The resulting patch-level embeddings are aggregated by MIL 350, which partitions patches into negative 360 and positive 370 bags based on whether they contain pathologically relevant information.

Imaging data 310 generally includes raw medical images taken by modalities including rotational Digital Subtraction Angiography (DSA), Magnetic Resonance Imaging (MRI), Magnetic Resonance Angiography (MRA), X-ray imaging, etc. The imaging data may include any suitable format, size, resolution, etc. The imaging data 310 includes relevant visual information (e.g., vascular structures, potential aneurysm risk symptoms, other relevant medical guidance, etc.) that may be used to determine features of an aneurysm.

Patch extraction 320 generally includes dividing the imaging data 310 into smaller, uniformly sized patches (e.g., a 128×128 image may be segmented into 16 total 32×32 pixel patches, 4 total 64×64 pixels, or other suitable patch dimensions). In some examples, with rotational and/or 3D image modalities, patch extraction 320 processes the imaging data 310 into a suitable format for the techniques described herein. Patch extraction 320 localizes analysis by breaking down the full image into smaller regions so that local tissue characteristics and subtle morphological variations (e.g., the irregular borders of an aneurysm) may be extracted as features.

Patch processing 330 generally extracts features from the patches using a Siamese attention driven feature mechanism. The present techniques efficiently group high-dimension features into concise lower-dimension feature embeddings for fusion (e.g., using convolutional layers, transformer-based encoders specialized for images, etc.). At patch processing 330, the low-level features (e.g., edges, textures, intensity gradients, etc.) of the extracted slices may be computed. This serves to abstract one or more patches into a representation emphasizing the clinically relevant signals while reducing noise.

Siamese attention 340 generally includes transforming one or more patches into a compact feature vector, a low-dimensional embedding that summarizes the salient visual properties of that patch. In one example, this representation includes using clustering techniques, including K-means clustering in a Siamese attention mechanism, K-nearest neighbors (KNN) algorithm to group and compare similar patch features. For example, Siamese attention 340 may include computing distances between patch feature vectors and then clustering similar patches together to form a representative embedding. Advantageously, this may help ensure that patches with similar anatomical structures (e.g., those showing a potential aneurysm region) are mapped closely together in the feature space while distinguishing them from patches with normal anatomy.

The Siamese attention 340 processes medical imaging (e.g., 3D rotational DSA/MRA scans) by dividing images into volumetric patches and dynamically grouping them into positive and negative bags via a multi-instance learning framework. In some examples, the present techniques may include self-attention mechanisms, which advantageously assign diagnostic relevance to image patches, isolating regions indicative of aneurysm rupture, rupture risk, aneurysm risk, etc. In some examples, the self-attention mechanism may identify morphology, like irregular borders or size discrepancies, with features derived from the imaging modality as spatially and rhythmically aligned with clinical risk factors (e.g., hypertension, smoking history, sedentary lifestyle, etc.), facilitating an integrated multi-modal assessment of aneurysm rupture probability.

MIL 350 generally includes a multi-instance learning framework for bagging. One or more complete imaging studies (or a specific slice/volume) may be treated as a “bag” comprised of numerous individual patch instances. The MIL 350 framework acknowledges that while many patches might not contain any pathological information, even a single positive patch is beneficial for diagnosis.

Negative bag 360 and positive bag 370 are generally the labeled bags from the MIL framework. Negative bag 360 may include patches that, after feature extraction, do not exhibit the characteristics of an aneurysm (e.g., smooth vessel walls, normal gradient patterns, etc.). Positive bag 370 may include one or more patches that show features (e.g., abnormal size, irregular shape, unusual contrast, etc.) which potentially indicate the presence of an aneurysm.

In some examples, the present techniques may include self-attention mechanisms which advantageously assign diagnostic relevance to image patches isolating regions indicative of aneurysm features (e.g., rupture imminent, rupture risk, etc.). This binary split between aneurysm features and no aneurysm features ensures that during training, the patches in positive bags might be used for further feature fusion and prediction, while the negative bags can be ignored or leveraged as background information.

FIG. 3B generally depicts an example hierarchical transformer image encoder method 380 (e.g., representing additional and/or alternative functionality of image feature extraction component 136 as described above, with respect to FIG. 1). Imaging data 310 is received by a patch tokenization operation 382, which partitions the imaging data into non-overlapping patches of fixed size and projects each patch into a token embedding of dimension do, forming an input sequence X_{img}∈ [symbol] ^{N×d}^{ₒ}, where N denotes the total number of patches. In some examples, with rotational and/or 3D image modalities, the patch tokenization operation 382 processes the volumetric imaging data 310 into a token sequence suitable for transformer-based processing. This tokenization localizes analysis by breaking down the full image into discrete spatial units so that both local tissue characteristics (e.g., the irregular borders of an aneurysm) and long-range contextual dependencies may be captured via self-attention.

The patch tokenization operation 382 provides the token sequence to a transformer encoder stack 384 that may process the token sequence through stacked multi-head self-attention and feed-forward layers, or other suitable hierarchical feature extraction operations, progressively aggregating local and global spatial dependencies across scales. At each layer, multi-head self-attention may allow each patch token to attend to all other tokens in the sequence, capturing both fine-grained local cues (e.g., edges, textures, intensity gradients, wall irregularities) and long-range contextual relationships (e.g., aneurysm extent relative to parent artery). The transformer encoder stack 384 outputs multi-level token representations 386, denoted

$${\{ {T}_{ℓ} \}}_{ℓ = 1}^{4} ,$$

shown as a side branch with a dashed arrow, indicating skip connections from each encoding stage for segmentation decoding. These multi-scale representations encode spatial features at progressively increasing levels of abstraction, enabling decoding to recover fine-grained boundary-level anatomical detail. The hierarchical nature of these representations may be beneficial for diagnosis because the representations may allow the segmentation decoder to selectively integrate features at the appropriate level of spatial resolution.

The transformer encoder stack 384 produces a bottleneck embedding 388, which encodes a holistic representation of the full input image from the final transformer layer. Global semantic context may be distilled by applying global average pooling followed by a nonlinear projection, resulting in a compact image-level content descriptor. For example, the content descriptor may be computed as:

$${h}_{img} = {ϕ}_{cnt}^{img} ( Pool ( {X}_{img} ) ) ∈ {\text{?}}^{{d}_{c}}$$

where

$${ϕ}_{cnt}^{img} ( \cdot )$$

denotes a nonlinear projection. The bottleneck embedding may be represented as b∈ [symbol] ^{N}^{_{b}}^{×d}^{_{b}}, where N_{b} denotes the number of bottleneck tokens and dy denotes the embedding dimension. In some examples, three parallel projection heads may process the bottleneck embedding 388: a severity projection head 390 that produces image-specific severity features, a segmentation projection head 392 that produces image-specific segmentation features, and a common projection head 394 that produces task-common features shared with the clinical modality. The severity projection head 390 encodes visual cues relevant to aneurysm rupture risk (e.g., wall irregularity, texture roughness, signal inhomogeneity). Although the projection heads are depicted as operating in parallel in FIG. 3B, it should be understood that additional and/or alternative configurations may also be used (e.g., sequential processing, selective activation of one or more projection heads, or other suitable arrangements).

For example, the three projection heads may produce:

$${s}_{sev}^{img} = {ϕ}_{sev}^{img} ( Pool ( b ) ) ∈ {\text{?}}^{{d}_{s}}$$

encoding visual cues relevant to aneurysm rupture risk,

$${s}_{seg}^{img} = {ϕ}_{seg}^{img} ( Pool ( b ) ) ∈ {\text{?}}^{{d}_{s}}$$

encoding spatial and structural cues relevant to anatomical delineation (e.g., vessel topology, lumen continuity, local contrast boundaries), and

$${z}_{com}^{img} = {ϕ}_{com}^{img} ( Pool ( b ) ) ∈ {\text{?}}^{{d}_{s}}$$

encoding features shared with the clinical modality (e.g., aneurysm size, neck width, parent artery) that contribute to both segmentation and rupture assessment. This disentangled factorization may ensure that the network learns functionally distinct yet semantically aligned representations for each downstream task.

### Example Multi-Modal Fusion and Feature Disentanglement

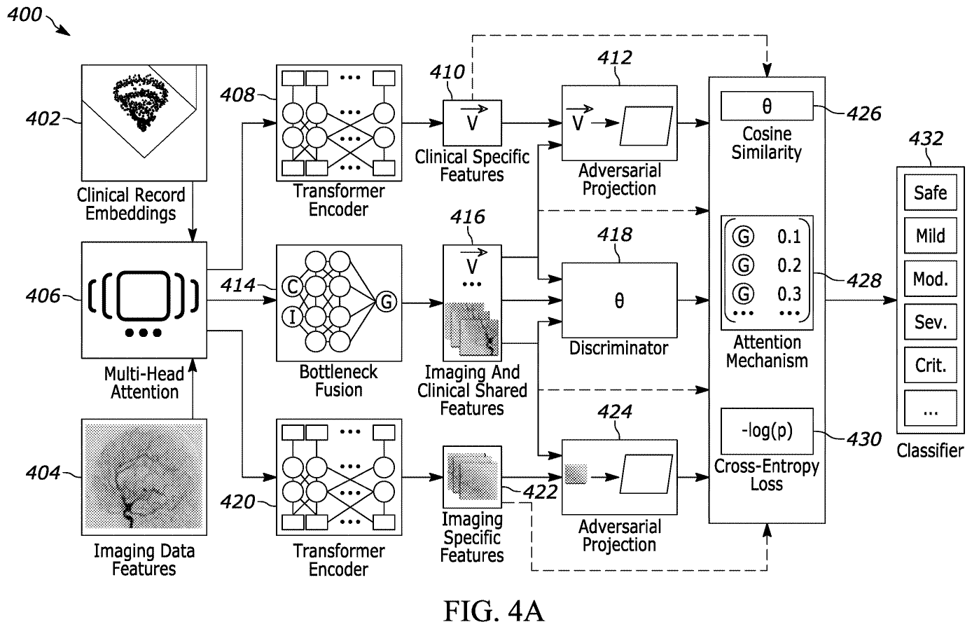

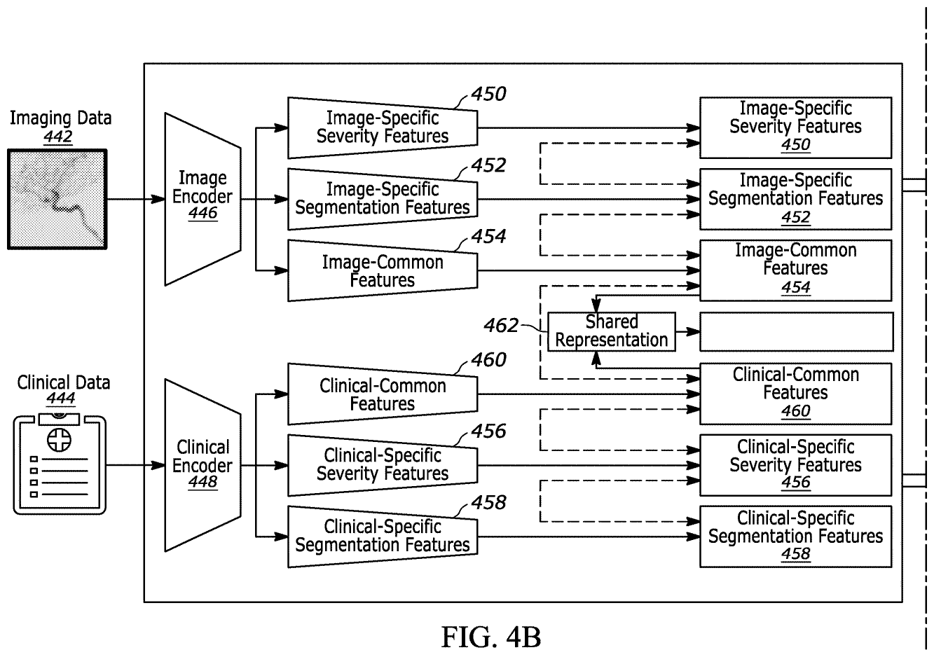

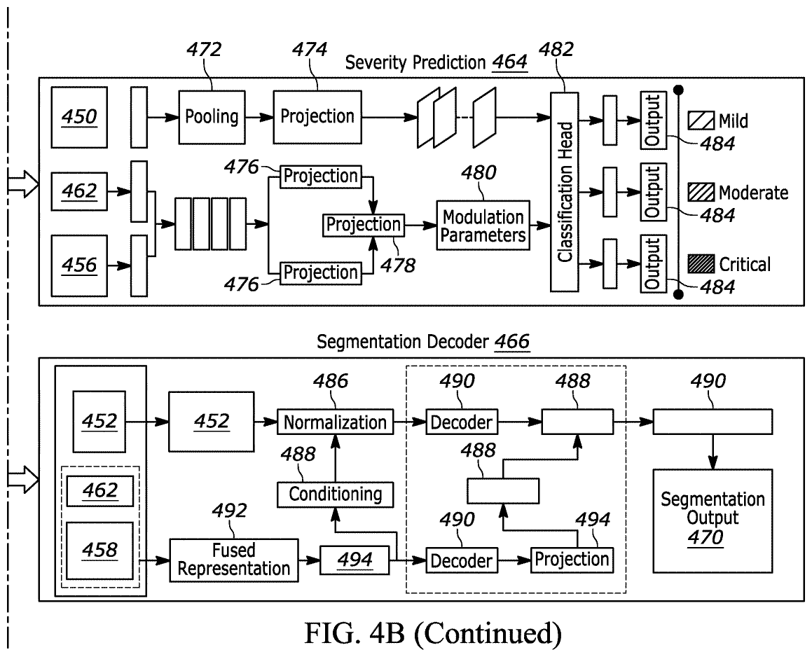

FIG. 4A generally depicts an example constrained modality-specific features (CMSF) block and logic flowchart 400. The flowchart 400 includes clinical record embeddings 402, imaging data features 404, multi-head attention 406, transformer encoder 408, clinical-specific features 410, adversarial projection 412, bottleneck fusion 414, imaging and clinical shared features 416, discriminator 418, transformer encoder 420, imaging-specific features 422, adversarial projection 424, cosine similarity 426, attention mechanism 428, cross-entropy loss 430, and classifier 432. The flowchart 400 generally depicts the processing paths for imaging and clinical data while emphasizing both shared (global) and modality-specific (local) feature extraction and their subsequent integration through attentional and adversarial mechanisms.

Clinical record embeddings 402 may be clinical input data (e.g., patient age, risk factors, medical history) that have been normalized and converted into a vector representation (e.g., through linear projection 260 and normalization 250). Medical imaging data features 404 may be imaging data (e.g., features extracted from rotational DSA, MRI, MRA, X-Ray, etc. modalities). The imaging data may have undergone patch-level inference using the techniques disclosed herein to form feature vectors.

Multi-head attention 406 is used for cross-modal fusion. After both clinical embeddings and imaging features (processed through patches and MIL) are individually prepared, they are merged into a multimodal feature embedding space using multi-head attention 406. Multi-head attention 406 enables the present techniques to simultaneously attend to different aspects of the input data across modalities. That is, one or more attention “heads” may focus on different relationships (e.g., correlating an anatomical measurement from an image with a clinical risk factor from text).

In some examples, the multi-head attention 406 may compute attention weights across multiple subspaces and then concatenate the outputs, to effectively align and fuse the disparate features from the imaging and clinical domains, thereby enabling cross-modal fusion and alignment of the imaging data features 404 and clinical record embeddings 402. Advantageously, the fusion of clinical and imaging information into a joint high-dimensional vector space enables greater accuracy, efficient use of resources, and interpretability.

Transformer encoder 408 may be for clinical-specific features 410 (local features). The transformer encoder focuses on distilling the independent aspects of the clinical data. Adversarial projection 412 (clinical features) projects these features into an adjusted latent space.

Bottleneck fusion 414 merges a portion of the multi-head attention 406 using a cross-modal attention mechanism to extract features that are modality-indiscriminate. Shared features 416 (global features) capture commonalities between the two modalities. The imaging and clinical shared features 416 are a unified representation, indicating that clinical and imaging modalities are aligned and fused.

Transformer encoder 420 and imaging-specific features 422 (local features) capture the detailed characteristics from the imaging modality. Modality discriminator 418 evaluates whether the incoming shared features truly represent modality-invariant data.

Cosine similarity 426 receives incoming vectors from both the adversarial projections and the shared features. This may help ensure that the local (modality-specific) features are orthogonal (i.e., non-redundant) relative to the global features. Attention mechanism 428 modulates the weighted contributions of various features based on their diagnostic relevance. Cross-entropy loss 430 signifies that misclassifications are penalized and guides the adversarial training process.

In some examples, such as that of FIG. 4A, the clinical-specific features 410, the shared features 416, and the imaging-specific features 422 may connect directly into the cosine similarity 426, attention mechanism 428, and cross-entropy loss 430. That is, a plurality of feature streams may contribute to the final loss calculation.

The classifier 432 classifies the final output: the aneurysm rupture probability classification, with risk categories such as Critical, Severe, Moderate, or Benign.

The present techniques employ adversarial projection 412, 424 and separate transformer encoder 408, 420 to learn both global and local features from the imaging and clinical modalities. The present techniques may employ a modality discriminator Θ, structured to compute a value:

∂=−0.5[log(Θ(X(sh)))+log(1−Θ(Y(sh)))]

such that during the discriminator phase, Θ attempts to distinguish the modality origin of the shared features while in the generation phase the backbone network may adapt to deceive Θ, thereby promoting the extraction of modality-invariant representations and enhancing cross-modal alignment.

In some examples, the bottleneck fusion 414 includes tokens {Ei∈R^{1×g}}ⱼ₌₁^{h} that facilitate knowledge exchange across modalities (e.g., medical imaging data and clinical records data). Cross-modal attention is directed through these bottleneck tokens E=[E1, . . . , Eh], providing a structured framework for representation. The transformer encoder update W_{q+1}=TE(W_{q}) is denoted as:

W_{q+1}=G+MLP(LN(G))

G=W_{q}+MSA(LN(W_{q}))

In these equations, Xₛₕ within R^{1×g} encapsulates features shared by clinical data and MRA, and Yₛₕ in R^{1×g} encapsulates features of clinical data influenced by MRA.

In further examples:

$$[ {Y}_{sh}^{q + 1} \vert \vert {Q}^{q + 1} \vert \vert {F}^{q + 1} ] = TE ( [ {Y}_{sh}^{q} \vert {Q}^{q} \vert \vert {F}^{q} ] ) ; {θ}_{y}$$

$$[ {X}_{sh}^{q + 1} \vert \vert {P}^{q + 1} \vert \vert {F}^{q + 1} ] = TE ( [ {X}_{sh}^{q} \vert {P}^{q} \vert \vert {F}^{q + 1} ] ) ; {θ}_{x}$$

where θ_{y} and θₓ represent parameters for the transformer encoder 408, 420. Further, MRA and clinical may share features through the shared space F of the two shared layers of the transformer.

In some examples, each layer, F is updated 2 times; first for imaging modality (e.g., imaging data features 404) and second for clinical data (e.g., clinical record embeddings 402). The number of shared tokens may be selected so that the total number of tokens per modality remains q<<m and q<<n.

In further examples, to ensure modality fusion learns global (e.g., shared across modalities as depicted in FIG. 4A as “G”) and local features (e.g., clinical-specific features 410, imaging-specific features 422) the present techniques include an adversarial projection 412, 424 to train a network.

For global feature learning (e.g., imaging, and clinical shared features 416), modal adaptation to exchange shared features includes a modality discriminator Θ (e.g., discriminator 418):

$$ϑ = - \frac{1}{2} [ log ( Θ ( {X}_{sh} ) ) + log ( 1 - Θ ( {y}_{sh} ) ) ]$$

The discriminator Θ may identify modality features. The backbone network generates features to deceive the discriminator ΘΘ. For modality or domain-specific features learning, Wₛₚ∈R^{1×k} extracts features from imaging data features 404 and Dₛₚ∈R^{1×k} for the clinical record embeddings 402 clinical data. These features are extracted using two layers of transformer encoder:

$$[ {D}_{sp}^{q + 1} \vert \vert {Q}^{q + 1} ] = TE ( [ {D}_{sp}^{q} \vert \vert {Q}^{q} ] )$$

$$[ {W}_{sp}^{q + 1} \vert \vert {P}^{q + 1} ] = TE ( [ {W}_{sp}^{q} \vert \vert {P}^{q} ] )$$

In some examples, the weights of the two transformer encoders 408, 420 are not shared, enabling the extraction of distinct and intrinsic features specific to one or more modalities in the local feature learning process. To ensure such, cosine similarity 426 minimizes the distance of Dₛₚ, Yₛₕ, Wₛₚ and Xₛₕ as:

$${L}_{cos - d} = max ( 0 , ( \frac{{D}_{sp} \cdot {Y}_{sh}}{\vert {D}_{sp} \vert \vert {Y}_{sh} \vert} ) )$$

$${L}_{cos - w} = max ( 0 , ( \frac{{W}_{sp} \cdot {X}_{sh}}{\vert {W}_{sp} \vert \vert {X}_{sh} \vert} ) )$$

As the cosine distance reduces towards zero, the cosine distance indicates that Dₛₚ and Wₛₚ are approaching orthogonality in an embedding space. One or more of these features may be considered local features independently related (e.g., unique) to their respective modalities (e.g., imaging data features 404, clinical record embeddings 402). The similarity decreasing toward zero reinforces that the learned features capture non-overlapping and independent information in the embedding space. This approach may enable minimization of feature redundancy while maintaining independent and discriminative representations for accurate analysis.

In some examples, to fully leverage the synergistic characteristic of the extracted features for the prediction of an aneurysm, an attention mechanism dynamically accumulates the features via a weighted combination, also including a fully connected layer for final prediction. In addition, the class-specific weighted cross-entropy may be measured between the input labels and the prediction.

By directly restraining the global and local features of the given two modalities to be efficient for the analysis, the present techniques generate predictions via fully connected layers and measure L_{pred} as:

L_{pred}=−[α₀(1−t)log(1−q)+α₁t log(q)]

Hence, this process enhances the performance of the discriminator by reducing the Lₘᵢₙ and increasing the performance of the modules by increasing Lₘₐₓ.

Lₘᵢₙ=L_{pred}+L_{cos-x}+L_{cos-y}-Lₘₛ and

Lₘₐₓ=−Lₘₛ,

The two independent transformer encoders 408, 420 may have unshared weights that are designed to extract modality-specific features; one branch extracts local features represented as Wₛₚ from MRA/DSA data, and the other branch extracts local features Dₛₚ from clinical data, according to the equations:

$$[ {W}_{sp}^{q + 1} \vert \vert {P}^{q + 1} ] = TE ( [ {W}_{sp}^{q} \vert \vert {P}^{q} ] ) and$$

$$[ {D}_{sp}^{q + 1} \vert \vert {Q}^{q + 1} ] = TE ( [ {D}_{sp}^{q} \vert \vert {Q}^{q} ] )$$

respectively. This enables the network to learn distinct and intrinsic features independent of one or more modalities while preserving information from both sources for subsequent fusion.

Finally, in some examples, the flowchart 400 may integrate the extracted features through an attention mechanism 428 that dynamically aggregates features via a weighted combination before passing the result through a fully connected layer for final prediction. The system may calculate a class-specific weighted cross-entropy loss:

L_{(pred)}=−[α₀(1−t)log(1−q)+α₁t log(q)]

and further optimizes the learning process through loss functions:

Lₘᵢₙ=L_{pred}+L_{cos-x}+L_{cos-y}-Lₘₛ and

Lₘₐₓ=−Lₘₛ,

thereby promoting a robust fusion of multi-modal features to support accurate intracranial aneurysm rupture severity prediction.

FIG. 4B generally depicts an example method 440 for adaptive disentangled multi-modal learning with patient-aware fusion for intracranial aneurysm segmentation and rupture risk prediction (e.g., representing additional and/or alternative functionality of multi-modal fusion component 137 and classification component 138 as described above, with respect to FIG. 1). The method 440 receives imaging data 442 and clinical data 444 as inputs. The imaging data 442 is processed by an image encoder 446, and the clinical data 444 is processed by a clinical encoder 448.

The image encoder 446 may produce three disentangled output representations: image-specific severity features 450, image-specific segmentation features 452, and image-common features 454. Similarly, the clinical encoder 448 may produce three disentangled output representations: clinical-specific severity features 456, clinical-specific segmentation features 458, and clinical-common features 460. A shared representation 462 may be derived from the image-common features 454 and clinical-common features 460, representing aligned cross-modal features shared between both modalities. Dashed arrows between the image encoder 446 and clinical encoder 448 outputs indicate contrastive alignment constraints that enforce semantic consistency between the corresponding image and clinical representations.

In severity prediction 464, the image-specific severity features 450 may be aggregated via a pooling 472 and projected through a projection 474 to form a compact severity representation. In parallel, the shared representation 462 and the clinical-specific severity features 456 may be projected through a projection 476 to produce patient-specific conditioning signals that encode both cross-modal context and clinical risk factors. These conditioning signals may be combined at a projection 478 to generate modulation parameters 480, which may adaptively scale and shift the severity features via Feature-wise Linear Modulation (FiLM) conditioning or other suitable feature-wise modulation technique.

The common features from both modalities may be fused via a cross-attention transformer block as:

$${z}_{fuse}^{com} = ρ ( [ {z}_{img}^{com} ; {z}_{clin}^{com} ] ) ∈ {ℝ}^{{d}_{f}}$$

where p(·) denotes a transformer-based fusion operator that captures interactions between population-level imaging characteristics and patient-specific clinical context. For example, the FILM conditioning vector may be computed as:

$${z}_{sev}^{cond} = ψ ( [ {s}_{sev}^{img} ; {s}_{sev}^{clin} ; {z}_{fuse}^{com} ] ) ∈ {ℝ}^{{d}_{f}}$$

where ψ(·) denotes a learnable projection that aggregates the severity-relevant inputs. The FiLM scaling and shifting parameters may then be produced by two learned linear generators:

$${γ}_{sev} = Γ ( {z}_{sev}^{cond} ) , {β}_{sev} = B ( {z}_{sev}^{cond} )$$

The FiLM-modulated severity representation may be computed as:

$${{s}_{\sim}}_{sev} = {s}_{sev}^{img} ⊙ ( 1 + {γ}_{sev} ) + {β}_{sev}$$

This may enable dynamic adaptation of feature representations for each patient. The modulated features may pass through a classification head 482 that produces output 484 in the form of a rupture severity classification 468 with categories including “Mild,” “Moderate,” and “Critical” although other categories may additionally and/or alternatively be included.

For example, the classification head 482 may estimate rupture severity probabilities as:

P_{\hat{ }cls}=softmax(φ_{cls}({tilde under (s)}ₛₑᵥ))∈Δ^{K-1}

where φ_{cls}(·) denotes a multi-layer perceptron with nonlinear activation, and Δ^{K-1} represents the (K−1)-dimensional probability simplex. By performing classification on FiLM-modulated severity features, the rupture prediction may be conditioned on patient-specific clinical risk factors while preserving the anatomical evidence encoded in imaging features.

In segmentation decoder 466, the image-specific segmentation features 452, the shared representation 462, and the clinical-specific segmentation features 458 may be combined into a fused representation 492. The fused representation 492 may pass through a projection 494 and then into a normalization operation 486. A conditioning 488 signal derived from the segmentation-relevant features modulates the normalized representation via Adaptive Instance Normalization (AdaIN) or other suitable adaptive normalization technique. The conditioned features may then pass through a final decoder 490 that progressively reconstructs the spatial output, producing a segmentation output 470 in the form of an aneurysm segmentation mask. For example, the AdaIN operation may normalize the fused representation by subtracting its channel-wise mean and dividing by its channel-wise standard deviation, then rescale and shift the result using the channel-wise mean and standard deviation computed from the segmentation-specific features:

$$AdaIN ( x , y ) = σ ( y ) \cdot \frac{x - μ ( x )}{σ ( x )} + μ ( y )$$

where x denotes the fused representation and y denotes the segmentation-specific conditioning features.

This patient-aware AdaIN conditioning may enable anatomically consistent decoding that adapts to patient-specific clinical and imaging cues. By aligning the channel-wise statistics of the fused representation with those of the image-specific segmentation features, AdaIN may inject clinically informed statistical priors into each stage of the segmentation decoder, improving spatial accuracy and boundary delineation of aneurysms. At each upsampling stage, skip connections

$${\{ {T}_{ℓ} \}}_{ℓ = 1}^{4}$$

from the corresponding image encoder are integrated to retain anatomical fidelity and preserve boundary-level information.

In some examples, such as that of FIG. 4B, severity prediction 464 and the segmentation decoder 466 may operate in parallel, sequentially, or in any other suitable configuration, each consuming the appropriate disentangled latent codes from the image encoder 446 and clinical encoder 448. That is, both FILM conditioning for severity prediction and AdaIN conditioning for segmentation decoding may simultaneously leverage the disentangled representations, enabling joint multi-task learning with shared feature disentanglement. This joint optimization may address the technical challenge of treating segmentation and risk prediction as independent tasks, by enabling complementary gradient signals from both tasks to flow through the shared representations during training, thereby improving both spatial localization accuracy and severity estimation performance.

The network may be trained end-to-end under a multi-task objective jointly supervising segmentation, rupture classification, and representation disentanglement. A segmentation loss combining Dice overlap and binary cross-entropy (BCE) terms may be adopted to accurately delineate aneurysm regions and handle class imbalance. A classification loss may guide rupture prediction using a standard cross-entropy loss applied to the predicted probability distribution over classes. An orthogonality regularization term (e.g., a penalty term added to the training objective that discourages correlation between feature subspaces) may minimize the correlation between modality-specific and shared subspaces within both image and clinical encoders, promoting disentangled latent factors that improve interpretability and reduce redundancy. A contrastive alignment loss based on the InfoNCE formulation may align semantic structure between modalities, pulling positive pairs (image-clinical common embeddings from the same patient) closer in the latent space while pushing apart embeddings from different patients. The overall training objective may integrate all components as a weighted sum, and the model may be optimized end-to-end using gradient backpropagation. For example, the total objective may be expressed as:

Lₜₒₜₐₗ=λ_{seg}L_{seg}+λ_{cls}L_{cls}+λₒᵣₜₕLₒᵣₜₕ+λ_{cont}L_{cont}

where the weighting coefficients A balance the contribution of each loss term. In some embodiments, other suitable contrastive loss formulations may be used in place of or in addition to InfoNCE.

For example, the InfoNCE loss may be computed as:

$${ℒ}_{cont} = - log \frac{exp ( sim ( {z}_{img}^{com} , {z}_{clin}^{com} ) / τ )}{{\sum }_{j = 1}^{N} exp sim ( {z}_{img}^{com} , {z}_{clin , j}^{com} ) / τ )}$$

where sim (·,·) denotes a similarity function such as cosine similarity, t is a temperature parameter, and the summation is over all samples in a batch including the positive pair and negative samples from different patients.

The orthogonality regularization loss may minimize the correlation between modality-specific and task-common subspaces to promote disentangled latent factors. For example, the orthogonality loss may be computed as:

$${ℒ}_{orth} = {‖ {S}_{img}^{T} {Z}_{img}^{com} ‖}_{F}^{2} + {‖ {S}_{clin}^{T} {Z}_{clin}^{com} ‖}_{F}^{2}$$

where S_{img} and S_{clin} denote the modality-specific feature matrices from the image and clinical encoders, respectively

$${Z}_{img}^{com} and {Z}_{clin}^{com}$$

denote the corresponding task-common feature matrices, and ∥ ·∥_{F} denotes the Frobenius norm. Minimizing this loss encourages the modality-specific and task-common subspaces to be orthogonal, thereby reducing feature redundancy and semantic leakage.

The segmentation loss may combine Dice overlap and binary cross-entropy (BCE) terms to accurately delineate aneurysm regions and handle class imbalance. For example, the Dice loss component may be computed as:

$${\text{?}}_{dice} = 1 - \frac{2 {\sum }_{i} {p}_{i} {g}_{i} + ϵ}{{\sum }_{i} {p}_{i} + {\sum }_{i} {g}_{i} + ϵ}$$

where pᵢ denotes the predicted probability at pixel i, gᵢ denotes the ground truth label at pixel i, and ∈ is a constant (e.g., small) for numerical stability. The Dice component measures region overlap between prediction and ground truth, while the BCE term provides pixel-wise supervision for boundary fidelity.

### Example Computer-Implemented Method

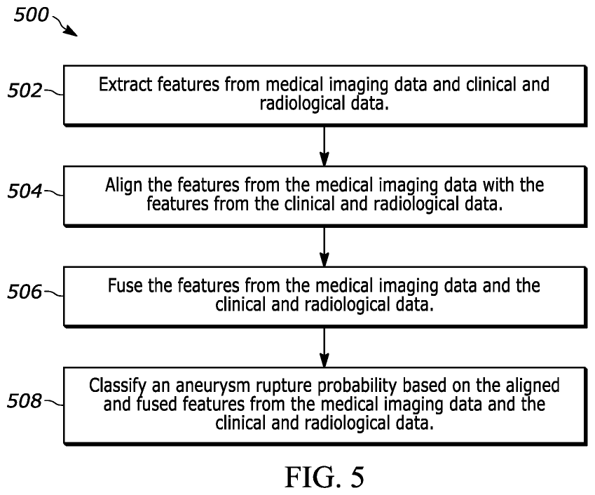

FIG. 5 depicts a computer-implemented method 500 for adaptive learning in multi-modal aneurysm rupture criticality analysis. In one example, the method 500 may include extracting features from medical imaging data and clinical and radiological data (block 502).

The method 500 may further include aligning the features from the medical imaging data with the features from the clinical and radiological data (block 504). For example, using a self-attention mechanism to correlate image-derived characteristics (such as aneurysm size, shape, and location) with clinical attributes (such as patient age, risk factors, or medical history).

The method 500 may further include fusing the features from the medical imaging data and the clinical and radiological data (block 506).

The method 500 may further include classifying an aneurysm rupture probability based on the aligned and fused features from the medical imaging data and the clinical and radiological data (block 508).

The method 500 may further include adversarial projection with a modality discriminator to enforce alignment between shared features and distinguishable modality-specific features. In some examples, the method 500 includes a modality discriminator configured to drive the extraction process so that features common to both the imaging and clinical data (e.g., aneurysm size) are effectively aligned, while characteristics (e.g., clinical risk factors like hypertension) are maintained separately. This may help ensure that the network learns robust representations for predicting rupture risk. Additionally or alternatively, the method 500 may include applying a loss function that enforces semantic consistency between shared feature representations across the medical imaging data and the clinical and radiological data.

The method 500 may further include applying orthogonality regularization and InfoNCE contrastive alignment to enforce meaningful disentanglement and cross-modal semantic coherence. In some examples, the method 500 employs orthogonality constraints to minimize correlation between modality-specific and common subspaces so that features common to both the imaging and clinical data (e.g., aneurysm size, neck width) are effectively aligned via contrastive learning, while modality-specific characteristics (e.g., imaging wall irregularity, clinical risk factors like hypertension) are preserved in separate non-overlapping subspaces. This may help ensure that the network learns robust and interpretable disentangled representations for predicting rupture risk. In some examples, the contrastive alignment may pull task-common representations from the medical imaging data and the clinical and radiological data for a same patient closer in a latent space while pushing apart task-common representations from different patients.

The method 500 may further comprise processing medical imaging data through a hierarchical transformer image encoder that partitions the input image into non-overlapping patch tokens and applies stacked multi-head self-attention layers to progressively aggregate local and global spatial dependencies. Such processing may enable the system to effectively capture both fine-grained anatomical cues (e.g., wall irregularity, vessel topology) and long-range contextual relationships (e.g., aneurysm extent relative to parent artery) while producing multi-level representations and a disentangled bottleneck embedding.

The method 500 may further include employing three dedicated projection heads on the image encoder bottleneck to decompose image representations into severity-specific, segmentation-specific, and task-common latent codes. In some examples, this disentanglement enables the network to robustly separate structural features relevant to boundary delineation (e.g., vessel topology, lumen continuity) from cues relevant to rupture risk assessment (e.g., wall irregularity, texture roughness), thus enhancing task-specific feature discrimination.

The method 500 may also further include computing a composite multi-task training objective combining segmentation loss, classification loss, orthogonality regularization, and InfoNCE contrastive alignment loss. This multi-task objective refines the learning process by jointly supervising segmentation, rupture classification, and representation disentanglement, encouraging robust and clinically generalizable feature integration.

The method 500 may further comprise decomposing features from each of the medical imaging data and the clinical and radiological data into modality-specific representations and task-common representations. In some examples, the modality-specific representations may capture information unique to each data source (e.g., imaging-specific wall irregularity features, clinical-specific patient history factors), while the task-common representations may capture shared semantic content relevant to both modalities (e.g., aneurysm size, morphological descriptors). The method 500 may further include enforcing orthogonality between the modality-specific representations and the task-common representations to reduce feature redundancy and semantic leakage between the representation subspaces.

The method 500 may further comprise processing medical imaging data through a hierarchical transformer image encoder. In some examples, the hierarchical transformer image encoder may partition the medical imaging data into non-overlapping patch tokens and process the patch tokens via stacked multi-head self-attention layers to produce multi-level spatial representations and a bottleneck embedding. The method 500 may further include decomposing the bottleneck embedding via one or more projection heads into at least severity-specific features, segmentation-specific features, and task-common features. This disentangled factorization may enable the network to learn functionally distinct yet semantically aligned representations for each downstream task.

The method 500 may further comprise processing clinical and radiological data through a token-based transformer clinical encoder. In some examples, each clinical variable may be embedded as a learnable token, and the token sequence may be processed through stacked self-attention and feed-forward layers to capture inter-variable correlations. The method 500 may further include decomposing the clinical representation via dedicated projection heads into severity-specific clinical features, segmentation-specific clinical features, and task-common clinical features shared with the image modality.

The method 500 may further comprise employing adaptive fusion mechanisms for integrating aligned clinical and imaging representations. In some examples, Feature-wise Linear Modulation (FILM) conditioning may generate adaptive scaling and shifting parameters from a conditioning vector that aggregates severity-relevant features from both modalities together with shared cross-modal representations, producing a patient-aware modulated representation for rupture severity prediction. Additionally or alternatively, Adaptive Instance Normalization (AdaIN) may condition a hierarchical segmentation decoder on segmentation-relevant features from both imaging and clinical modalities, enabling anatomically consistent spatial reconstruction that adapts to patient-specific clinical and imaging context.

The method 500 may include performing patch-level inference on the medical imaging data to divide images into contiguous patches that form at least one of positive or negative bags based on the presence or absence of aneurysm features. For instance, patches that depict abnormal vessel morphology or irregular aneurysm borders may be grouped as positive bags, whereas patches without aneurysm indications may form negative bags.

The method 500 may further process the medical imaging data (e.g., MRA/DSA images) by extracting features corresponding to an aneurysm's size, shape, or location, while processing clinical data comprising patient age, risk-associated factors, or medical history. This dual-modality approach may help ensure that both visual and contextual patient information are available for subsequent analysis.

The method 500 may additionally include converting clinical data into embeddings via at least one of a linear projection or a learnable token embedding. For example, raw numerical or categorical clinical values (e.g., a binary code representing gender or a numerical age value) may be projected into a high-dimensional vector space to form a consistent set of embeddings for integration with imaging features.

The method 500 may further comprise performing a patch-level inference on image segments that exhibit aneurysm features and employing at least one of K-means clustering or a Siamese attention mechanism to embed the medical imaging data into a low-level feature representation. Such processing may enable the system to effectively focus on salient regions while reducing the dimensionality of the imaging data.

The method 500 may further include employing a Siamese attention mechanism that extracts and categorizes features from the image patches into group-level representations. In some examples, grouping enables the network to robustly cluster similar patterns (e.g., consistently irregular aneurysm margins), thus enhancing feature discrimination.

The method 500 may further include using transformer encoders to process both image-derived features and clinical data embeddings. This facilitates the extraction of complex relationships across the modalities by enabling long-range dependencies to be captured.

The transformer encoder may integrate global and local features using at least one of multi-head self-attention, a multi-layer perceptron, residual connections, or normalization layers. For example, multi-head self-attention may capture global contextual cues from an overall aneurysm region, while a multi-layer perceptron refines detailed local structures from the patches.

The method 500 may further comprise fusing the features from the medical imaging data and the clinical data embeddings through a bottleneck that combines common features. In examples, this bottleneck is intended to restrict and focus the fusion, ensuring that shared information is passed forward.

The bottleneck may include cross-modal attention mechanisms that facilitate knowledge exchange between the medical imaging data and the clinical data embeddings. For instance, the cross-modal attention layer enables features such as the size of an aneurysm observed in an image to be directly contrasted and correlated with a corresponding clinical risk factor.

The method 500 may further include classifying the aneurysm rupture probability into at least one of the categories of: critical, severe, moderate, or benign. In some examples, the explicit classification into these risk categories supports clinicians in making informed treatment decisions based on the predicted rupture risk.

The method 500 may also further include performing adversarial projection with a modality discriminator to compute a loss based on logarithmic evaluations of the shared features from the clinical data and the medical imaging data. This additional step refines the learning process by penalizing misaligned representations and encouraging robust feature integration. Additionally or alternatively, the method 500 may include computing a loss via a training objective that jointly supervises segmentation and classification while regularizing learned representations.

Overall, through the coordinated application of one or more of transformer-based processing, multi-instance learning with patch-level inference, Siamese attention grouping, linear projection embedding of clinical data, and adversarial projection with modality discrimination, hierarchical transformer image encoding, token-based transformer clinical encoding, disentangled representation learning with modality-specific and task-common projection heads, FiLM-based patient-aware severity prediction, AdaIN-guided anatomically consistent segmentation decoding, and a multi-task objective combining segmentation, classification, orthogonality, and contrastive alignment losses, the method 500 enables adaptive learning across medical imaging and clinical data to predict aneurysm rupture probability with enhanced accuracy and interpretability.

### Example Feature Representation Visualization

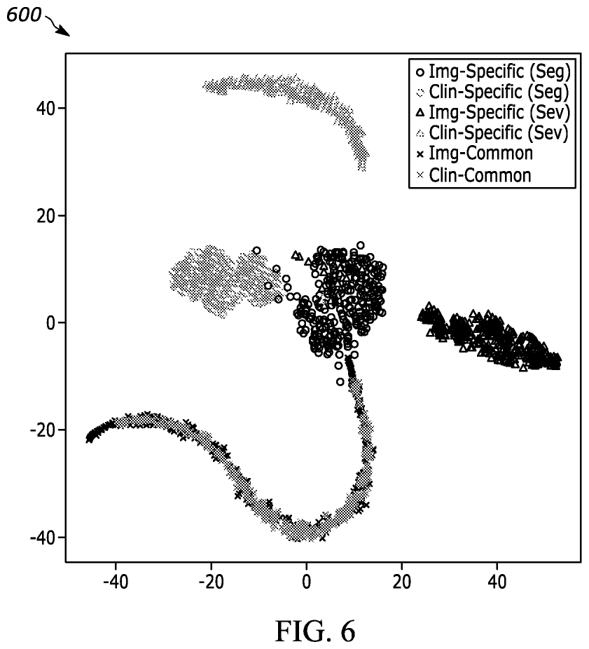

FIG. 6 depicts a visualization 600 of task-specific and common feature representations for both segmentation and severity prediction tasks across image and clinical modalities. The visualization 600 shows a two-dimensional embedding space generated using t-distributed Stochastic Neighbor Embedding (t-SNE), illustrating the spatial distribution of the disentangled latent representations produced by the method 440 as described above, with respect to FIG. 4B (e.g., representing functionality of multi-modal fusion component 137 and classification component 138 as described above, with respect to FIG. 1).

The visualization 600 includes six feature categories corresponding to the disentangled outputs of the image encoder 446 and clinical encoder 448. Image-specific features for segmentation (corresponding to image-specific segmentation features 452) and clinical-specific features for segmentation (corresponding to clinical-specific segmentation features 458) are shown as separate clusters, indicating that the modality-specific segmentation representations capture distinct information from each modality. Image-specific features for severity prediction (corresponding to image-specific severity features 450) and clinical-specific features for severity prediction (corresponding to clinical-specific severity features 456) are similarly shown as separate clusters.

The common features derived from both image and clinical modalities (corresponding to image-common features 454 and clinical-common features 460) exhibit overlap in a central region of the visualization 600, forming a well-aligned cluster. This overlap indicates successful multimodal feature integration via contrastive alignment, indicating that the shared representation 462 captures patient-level information that is semantically consistent across both modalities.

In contrast, the modality-specific representations for segmentation and severity prediction remain separated from each other and from the shared feature space. This structured separation may demonstrate the ability of the framework to preserve task-specific and modality-specific information while simultaneously learning a unified representation for multimodal reasoning. The disentanglement visible in the visualization 600 may confirm that the orthogonality constraints and contrastive alignment losses described with respect to FIG. 4B may reduce semantic leakage and feature redundancy between the modality-specific and shared subspaces.

### Example Aspects

Example 1. A computer-implemented method for adaptive learning in multi-modal aneurysm rupture criticality analysis comprising: extracting, by one or more processors, features from medical imaging data and clinical and radiological data; aligning, by the one or more processors, the features from the medical imaging data with the features from the clinical and radiological data; fusing, by the one or more processors, the features from the medical imaging data and the clinical and radiological data; and classifying, by the one or more processors, an aneurysm rupture probability based on the aligned and fused features from the medical imaging data and the clinical and radiological data.

Example 2. The computer-implemented method of example 1, wherein classifying an aneurysm rupture probability includes enforcing alignment between modality-specific features and shared features by at least one of: (i) an adversarial projection with a modality discriminator that distinguishes between shared features common to both modalities and modality-specific features independent to one or more modalities, or (ii) a loss function that enforces semantic consistency between shared feature representations across the medical imaging data and the clinical and radiological data.

Example 3. The computer-implemented method of example 1, wherein extracting features from medical imaging data includes at least one of positive or negative bags based on aneurysm features from patch-level inferences of images processed into patches.

Example 4. The computer-implemented method of example 1, wherein extracting features from the clinical and radiological data includes converting the clinical and radiological data into embeddings by at least one of a linear projection or a learnable token embedding.

Example 5. The computer-implemented method of example 1, wherein extracting features from the medical imaging data includes: (i) patch-level inferencing on segments of slices exhibiting aneurysm features, and (ii) embedding the medical imaging data into patches in a low-level embedded feature representation by at least one of a K-means clustering or a Siamese attention mechanism, wherein the feature representation is at least one of extracting or categorizing group-level representations, group features, or individual features from patches exhibiting an aneurysm.

Example 6. The computer-implemented method of example 1, wherein extracting features includes: processing medical image-derived features and clinical and radiological data embeddings, and integrating global and local features via a self-attention transformer encoder by at least one of: (i) multi-head self-attention, (ii) a multi-layer perceptron, (iii) residual connections, or (iv) normalization layers.

Example 7. The computer-implemented method of example 1, wherein fusing the features includes: combining common features from the medical imaging data and clinical and radiological data embeddings, and exchanging knowledge between the medical imaging and clinical and radiological data embeddings by cross-modal attention.

Example 8. The computer-implemented method of example 1, wherein classifying the aneurysm rupture probability includes computing a loss based on logarithmic evaluations of shared features from the clinical and radiological data and the medical image data by an adversarial projection with a modality discriminator.

Example 9. The computer-implemented method of example 1, wherein the medical imaging data is at least one of magnetic resonance angiography (MRA) or digital subtraction angiography (DSA), the medical imaging data includes at least one of an aneurysm (i) size, (ii) shape, or (iii) location, and the clinical and radiological data includes at least one of a patient (i) age, (ii) risk-associated factors, or (iii) medical history.

Example 10. The computer-implemented method of any one of Examples 1-9, wherein extracting features comprises decomposing features from one or more of the medical imaging data and the clinical and radiological data into modality-specific representations and task-common representations, and enforcing orthogonality between the modality-specific representations and the task-common representations.

Example 11. The computer-implemented method of any one of Examples 1-10, wherein extracting features from the medical imaging data comprises: partitioning the medical imaging data into non-overlapping patch tokens; processing the patch tokens via stacked multi-head self-attention layers to produce multi-level spatial representations and a bottleneck embedding; and decomposing the bottleneck embedding via one or more projection heads into at least severity-specific features, segmentation-specific features, and task-common features.

Example 12. The computer-implemented method of any one of Examples 1-11, wherein aligning the features comprises applying a contrastive alignment loss that pulls task-common representations from the medical imaging data and the clinical and radiological data for a same patient closer in a latent space while pushing apart task-common representations from different patients.

Example 13. The computer-implemented method of any one of Examples 1-12, wherein fusing the features comprises generating adaptive scaling and shifting parameters from a conditioning vector aggregating severity-relevant features from both modalities and shared cross-modal representations, and modulating a severity feature representation using Feature-wise Linear Modulation (FILM) conditioning to produce a patient-aware representation for rupture severity prediction.

Example 14. The computer-implemented method of any one of Examples 1-13, wherein fusing the features comprises conditioning a segmentation decoder on segmentation-relevant features from both the medical imaging data and the clinical and radiological data via Adaptive Instance Normalization (AdaIN), enabling anatomically consistent spatial reconstruction that adapts to patient-specific context.

Example 15. A computing system comprising: one or more processors; and one or more memories storing computer-executable instructions that cause the one or more processors to perform operations comprising any one of Examples 1-14.

Example 16. One or more non-transitory computer-readable media storing computer-executable instructions that, when executed by one or more processors, cause the one or more processors to perform operations comprising any one of Examples 1-14.

Example 17. Any one of Examples 1-16, wherein applying adversarial projection by directing shared features from clinical data and MRA images through a modality discriminator.

Example 18. Any one of Examples 1-16, wherein processing the clinical data by applying a linear transformation D to one or more clinical attributes, adding a learned position encoding B, and forming a clinical attribute matrix A.

Example 19. Any one of Examples 1-16, wherein iteratively updating global shared and local feature tokens via transformer encoder operations, wherein the transformer encoder updates are performed twice per layer for one or more of the MRA and clinical modalities.

Example 20. Any one of Examples 1-16, wherein reducing the number of shared tokens such that the total number of tokens per modality satisfies q<<m and q<<n.

Example 21. Any one of Examples 1-16, wherein applying an adversarial loss function computed as negative half the sum of the logarithm of the modality discriminator output on the shared MRA features and the logarithm of one minus the modality discriminator output on the shared clinical features.

Example 22. Any one of Examples 1-16, wherein the fused global and local features are obtained via an attention mechanism followed by a fully connected layer to produce the final prediction output.

Example 23. Any one of Examples 1-16, wherein embedding the clinical data into a lower-dimension feature representation through normalization, a linear projection, and learned position encoding, and wherein extracting patch-level features from the MRA image data via a Siamese attention grouping module.

Example 24. Any one of Examples 1-16, wherein performing global multi-modal fusion by integrating clinical and MRA image features using a transformer encoder configured with multi-head self-attention, residual connections, and bottleneck tokens.

Example 25. Any one of Examples 1-16, wherein applying an adversarial projection mechanism with a modality discriminator to align shared features and distinguish modality-specific features.

Example 26. Any one of Examples 1-16, wherein updating shared and modality-specific features iteratively via a transformer encoder using independent parameter sets for clinical and MRA modalities.

Example 27. Any one of Examples 1-16, wherein a cosine similarity loss function is used to minimize the distance between modality-specific feature vectors and the corresponding shared feature vectors, thereby promoting orthogonality in the embedding space.

Example 28. Any one of Examples 1-16, wherein computing a composite loss comprises at least one of a class-specific weighted cross-entropy loss, cosine similarity losses, or an adversarial loss, wherein reduction of the composite loss enhances the performance of the multi-modal feature fusion system.

### Additional Considerations

Some of the figures described herein illustrate example block diagrams having one or more functional components. It will be understood that such block diagrams are for illustrative purposes and the devices described and shown may have additional, fewer, or alternate components than those illustrated. Additionally, in various aspects, the components (as well as the functionality provided by the respective components) may be associated with or otherwise integrated as part of any suitable components.

Some aspects of the disclosure relate to a non-transitory computer-readable storage medium having instructions/computer-readable storage medium thereon for performing various computer-implemented operations. The term “instructions/computer-readable storage medium” is used herein to include any medium that is capable of storing or encoding a sequence of instructions or computer codes for performing the operations, methodologies, and techniques described herein. The media and computer code may be those specially designed and constructed for the purposes of the aspects of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an aspect of the disclosure may be implemented using Python, R, MATLAB, Julia, SAS, Java, C++, or other object-oriented programming languages and development tools. Additional examples of computer code include encrypted code and compressed code. Moreover, an aspect of the disclosure may be downloaded as a computer program product, which may be transferred from a remote computer (e.g., a server computer) to a requesting computer (e.g., a client computer or a different server computer) via a transmission channel. Another aspect of the disclosure may be implemented in hardwired circuitry in place of, or in combination with, machine-executable software instructions.

The use herein of “a” or “an” are employed to describe elements and components of the examples herein. This is done merely for convenience and to give a general sense of the description. This description, and the claims that follow, should be read to include one or at least one and the singular also includes the plural unless expressly stated or it is obvious that it is meant otherwise. As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the terms “approximately,” “substantially,” “substantial,” “roughly,” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to +10% of that numerical value, such as less than or equal to +5%, less than or equal to +4%, less than or equal to +3%, less than or equal to +2%, less than or equal to #1%, less than or equal to +0.5%, less than or equal to +0.1%, or less than or equal to +0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to #1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

What is claimed is:

1. A computer-implemented method for adaptive learning in multi-modal aneurysm rupture criticality analysis comprising:
extracting, by one or more processors, features from medical imaging data and clinical and radiological data;
aligning, by the one or more processors, the features from the medical imaging data with the features from the clinical and radiological data;
fusing, by the one or more processors, the features from the medical imaging data and the clinical and radiological data; and
classifying, by the one or more processors, an aneurysm rupture probability based on the aligned and fused features from the medical imaging data and the clinical and radiological data.

2. The computer-implemented method of claim 1, wherein classifying an aneurysm rupture probability includes enforcing alignment between modality-specific features and shared features by at least one of: (i) an adversarial projection with a modality discriminator that distinguishes between shared features common to both modalities and modality-specific features independent to one or more modalities, or (ii) a loss function that enforces semantic consistency between shared feature representations across the medical imaging data and the clinical and radiological data.

3. The computer-implemented method of claim 1, wherein extracting features includes decomposing features from one or more of the medical imaging data and the clinical and radiological data into modality-specific representations and task-common representations, and enforcing orthogonality between the modality-specific representations and the task-common representations.

4. The computer-implemented method of claim 1, wherein extracting features from the clinical and radiological data includes converting the clinical and radiological data into embeddings by at least one of a linear projection or a learnable token embedding.

5. The computer-implemented method of claim 1, wherein extracting features from the medical imaging data includes:
partitioning the medical imaging data into non-overlapping patch tokens;
processing the patch tokens via stacked multi-head self-attention layers to produce multi-level spatial representations and a bottleneck embedding; and
decomposing the bottleneck embedding via one or more projection heads into at least severity-specific features, segmentation-specific features, and task-common features.

6. The computer-implemented method of claim 1, wherein extracting features includes:
processing medical image-derived features and clinical and radiological data embeddings, and
integrating global and local features via a self-attention transformer encoder by at least one of:
(1) multi-head self-attention,
(2) a multi-layer perceptron,
(3) residual connections, or
(4) normalization layers.

7. The computer-implemented method of claim 1, wherein fusing the features includes:
combining common features from the medical imaging data and clinical and radiological data embeddings, and
exchanging knowledge between the medical imaging and clinical and radiological data embeddings by cross-modal attention.

8. The computer-implemented method of claim 1, wherein classifying the aneurysm rupture probability includes computing a loss that penalizes misalignment between shared features and modality-specific features from the clinical and radiological data and the medical imaging data by at least one of: (i) logarithmic evaluations via an adversarial projection with a modality discriminator, or (ii) a training objective that jointly supervises segmentation and classification while regularizing learned representations.

9. The computer-implemented method of claim 1, wherein the medical imaging data is at least one of magnetic resonance angiography (MRA) or digital subtraction angiography (DSA), the medical imaging data includes at least one of an aneurysm (i) size, (ii) shape, or (iii) location, and the clinical and radiological data includes at least one of a patient (i) age, (ii) risk-associated factors, or (iii) medical history.

10. A computing system comprising:
one or more processors; and
one or more memories storing computer-executable instructions that cause the one or more processors to at least:
extract features from medical imaging data and clinical and radiological data;
align the features from the medical imaging data with the features from the clinical and radiological data;
fuse the features from the medical imaging data and the clinical and radiological data; and
classify an aneurysm rupture probability based on the aligned and fused features from the medical imaging data and the clinical and radiological data.

11. The computing system of claim 10, wherein classifying an aneurysm rupture probability includes enforcing alignment between modality-specific features and shared features by at least one of: (i) an adversarial projection with a modality discriminator that distinguishes between shared features common to both modalities and modality-specific features independent to one or more modalities, or (ii) a loss function that enforces semantic consistency between shared feature representations across the medical imaging data and the clinical and radiological data.

12. The computing system of claim 10, wherein extracting features includes decomposing features from one or more of the medical imaging data and the clinical and radiological data into modality-specific representations and task-common representations, and enforcing orthogonality between the modality-specific representations and the task-common representations.

13. The computing system of claim 10, wherein extracting features from the clinical and radiological data includes converting the clinical and radiological data into embeddings by at least one of a linear projection or a learnable token embedding.

14. The computing system of claim 10, wherein extracting features from the medical imaging data includes:
partitioning the medical imaging data into non-overlapping patch tokens;
processing the patch tokens via stacked multi-head self-attention layers to produce multi-level spatial representations and a bottleneck embedding; and
decomposing the bottleneck embedding via one or more projection heads into at least severity-specific features, segmentation-specific features, and task-common features.

15. The computing system of claim 10, wherein extracting features includes:
processing medical image-derived features and clinical and radiological data embeddings, and
integrating global and local features via a self-attention transformer encoder by at least one of:
(1) multi-head self-attention,
(2) a multi-layer perceptron,
(3) residual connections, or
(4) normalization layers.

16. The computing system of claim 10, wherein fusing the features includes:
combining common features from the medical imaging data and clinical and radiological data embeddings, and
exchanging knowledge between the medical imaging and clinical and radiological data embeddings by cross-modal attention.

17. The computing system of claim 10, wherein classifying the aneurysm rupture probability includes computing a loss that penalizes misalignment between shared features and modality-specific features from the clinical and radiological data and the medical imaging data by at least one of: (i) logarithmic evaluations via an adversarial projection with a modality discriminator, or (ii) a training objective that jointly supervises segmentation and classification while regularizing learned representations.

18. The computing system of claim 10, wherein the medical imaging data is at least one of magnetic resonance angiography (MRA) or digital subtraction angiography (DSA), the medical imaging data includes at least one of an aneurysm (i) size, (ii) shape, or (iii) location, and the clinical and radiological data includes at least one of a patient (i) age, (ii) risk-associated factors, or (iii) medical history.

19. One or more non-transitory computer-readable media storing computer-executable instructions that, when executed by one or more processors, cause the one or more processors to perform operations comprising:
extracting features from medical imaging data and clinical and radiological data;
aligning the features from the medical imaging data with the features from the clinical and radiological data;
fusing the features from the medical imaging data and the clinical and radiological data; and
classifying an aneurysm rupture probability based on the aligned and fused features from the medical imaging data and the clinical and radiological data.

20. The one or more non-transitory computer-readable media of claim 19, wherein classifying an aneurysm rupture probability includes enforcing alignment between modality-specific features and shared features by at least one of: (i) an adversarial projection with a modality discriminator that distinguishes between shared features common to both modalities and modality-specific features independent to one or more modalities, or (ii) a loss function that enforces semantic consistency between shared feature representations across the medical imaging data and the clinical and radiological data.
